(19) United States
(12) Patent Application Publication
(10) Pub. No.: US 2025/0235340 A1
(43) Pub. Date: Jul. 24, 2025

(54) MY Shield

(71) Applicants: Katrina L. Royster, Germantown, MD (US); Shelley A. Walker, Montgomery Village, MD (US)

(72) Inventors: Katrina L. Royster, Germantown, MD (US); Shelley A. Walker, Montgomery Village, MD (US)

(21) Appl. No.: 18/418,157

(22) Filed: Jan. 19, 2024

(51) Int. Cl. A61F 5/00

(52) U.S. Cl. CPC A61F 5/0096

(57) ABSTRACT

The claimed invention is an up-to-date alternative for a rape device. We would like to make a claim to have the invention patented and make it available commercially. The invention is put together by measured galvanized metal that has been sanded and smoothed down on the front of the metal, on the rear of the metal, and around the edges of the metal. A rubber strip containing a safe adhesive to seal a rubber strip to go around the edge of the metal. My Shield will be made in three different sizes but not limited to the three sizes. It's important to get this patented because it's a simple invention and it provides an up-to-date option to creating a rape device for women, men, and children.

My Shield

### BRIEF DESCRIPTION OF THE DRAWINGS

### DRAWINGS

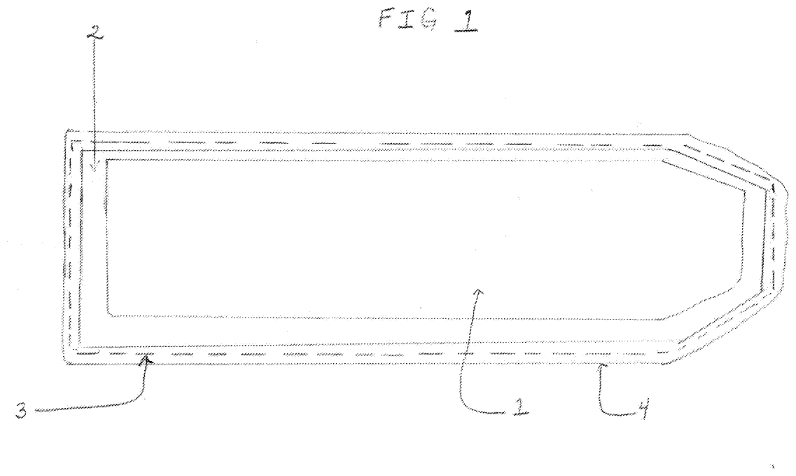

FIG. 1 is a perspective view of My Shield showing our design in a small size design.

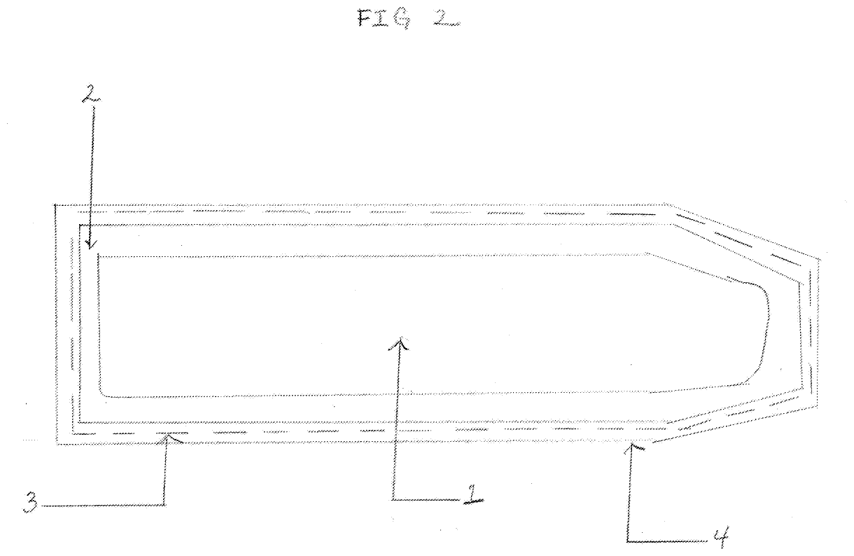

FIG. 2 is a perspective view of My Shield showing our design in a medium size design.

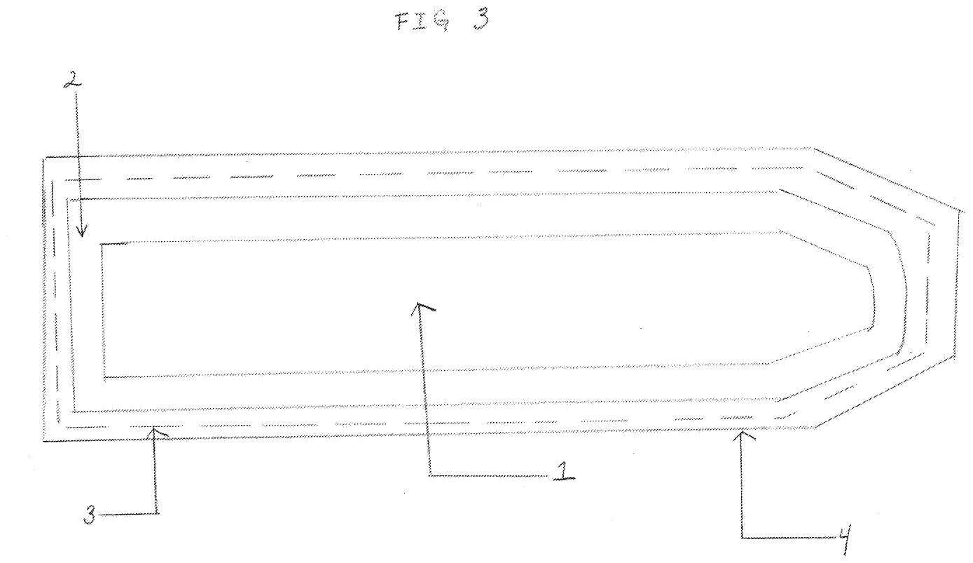

FIG. 3 is a perspective view of My Shield showing our design in a large size design.

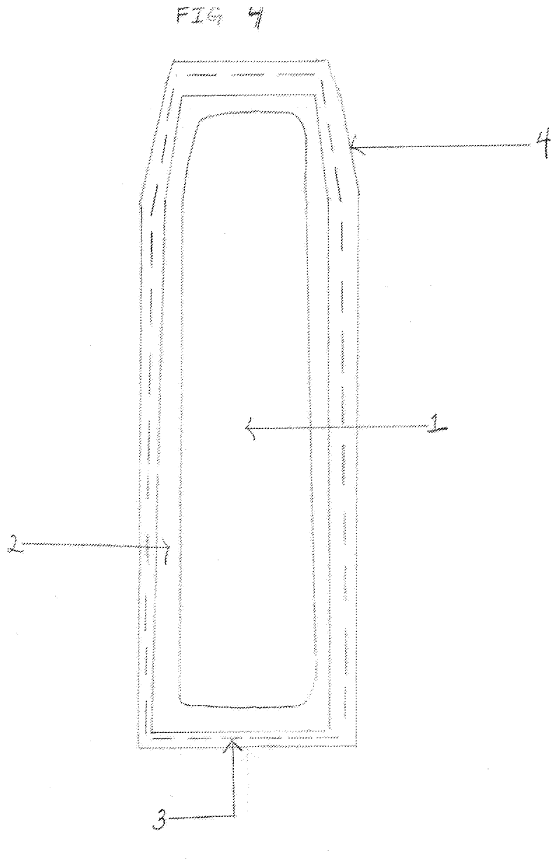

FIG. 4 is a frontal elevational view of the small size thereof.

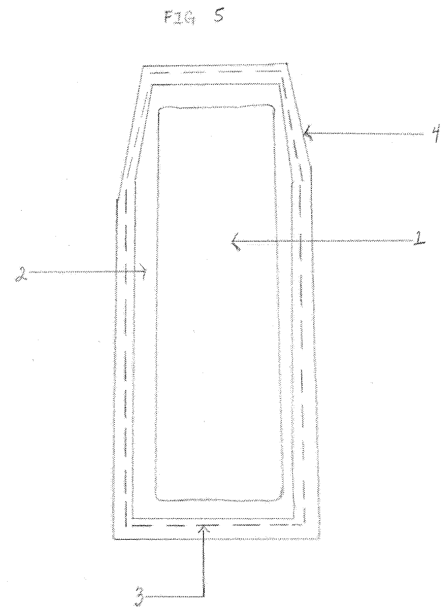

FIG. 5 is a frontal elevational view of the medium size thereof.

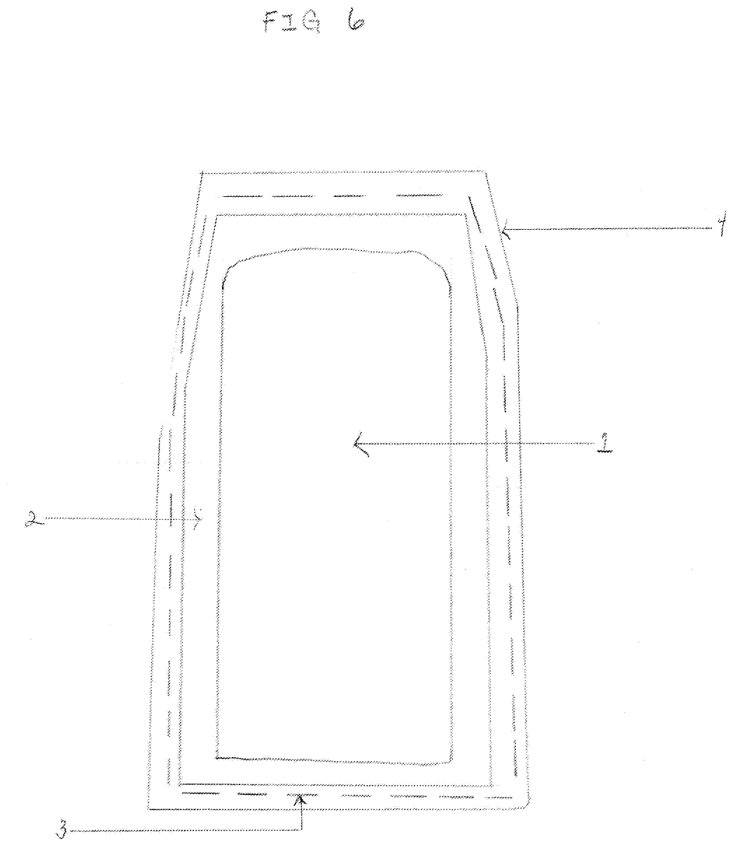

FIG. 6 is a frontal elevational view of the large size thereof.

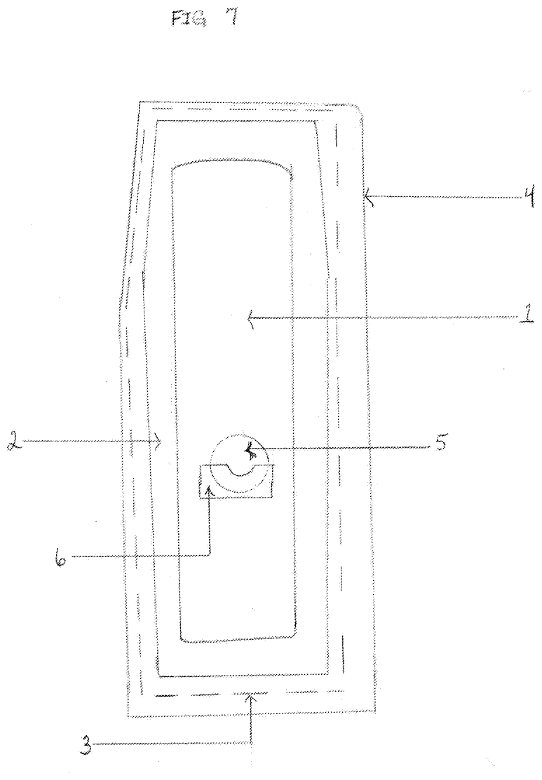

FIG. 7 is a rear elevational view of the small size with a sharp object thereof.

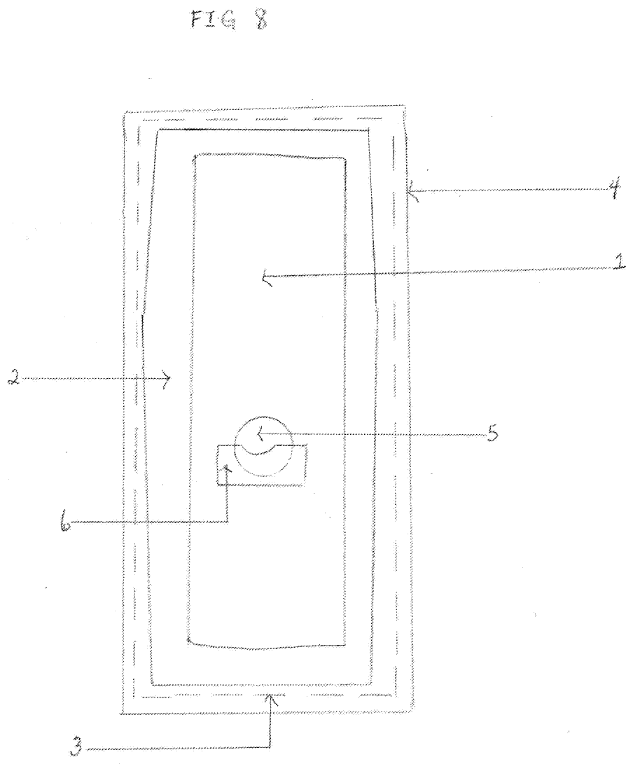

FIG. 8 is a rear elevational view of the medium size with a sharp object thereof.

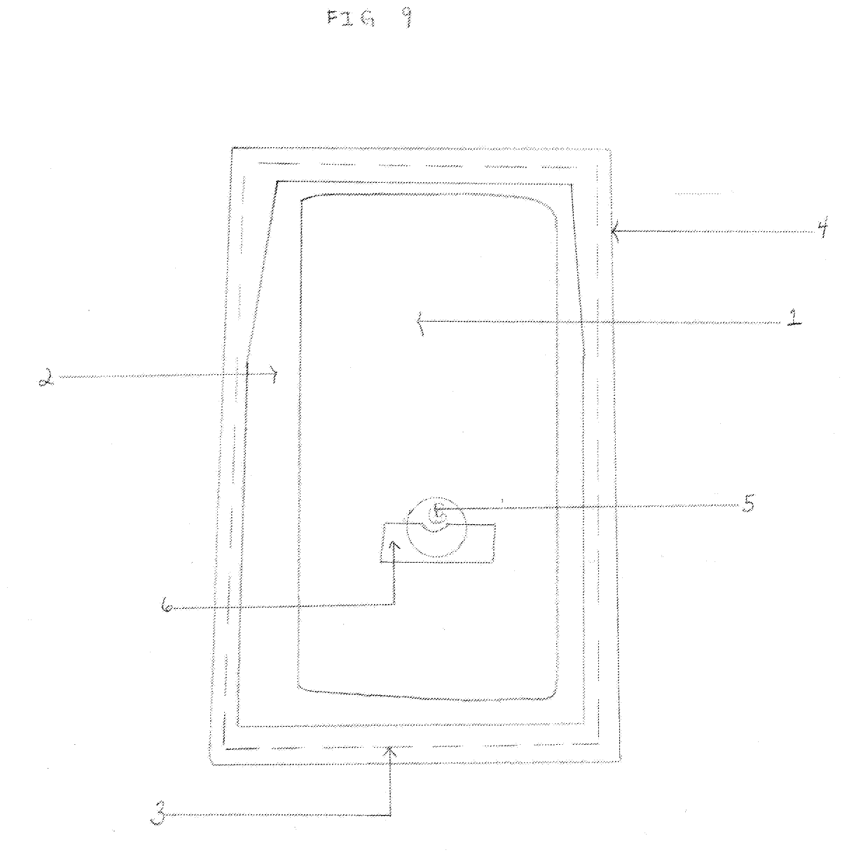

FIG. 9 is a rear elevational view of the large size with a sharp object thereof.

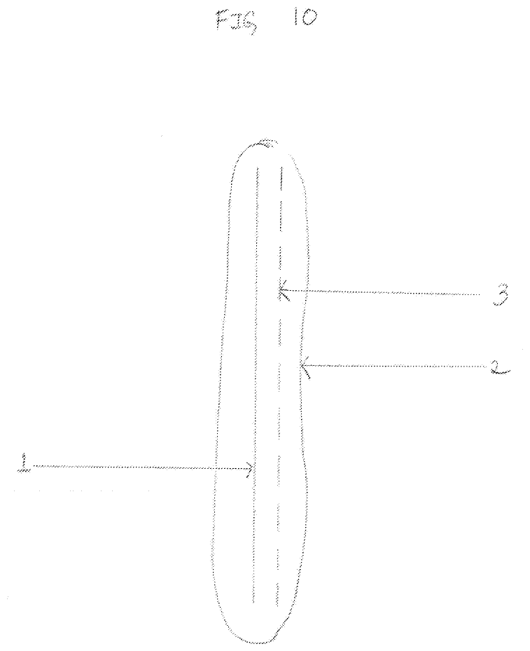

FIG. 10 is a side elevational view of the small, medium, and large; the opposite side being a mirror image thereof.

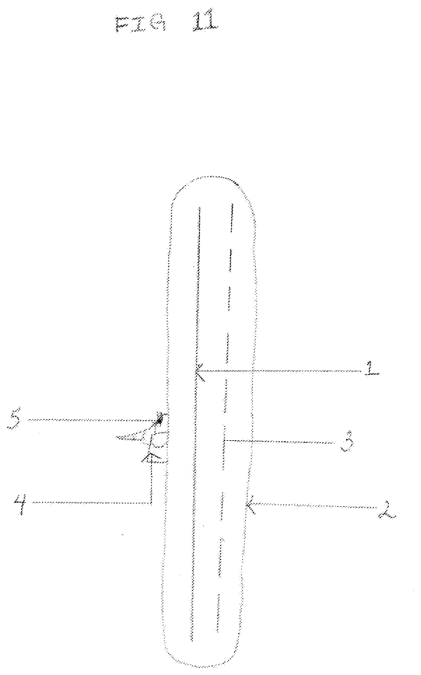

FIG. 11 is a side elevational view of the small, medium, and large with a sharp object thereof.

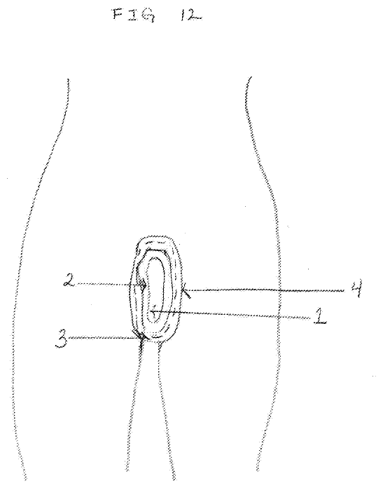

FIG. 12 is a frontal view that illustrates how a person can use the invention.

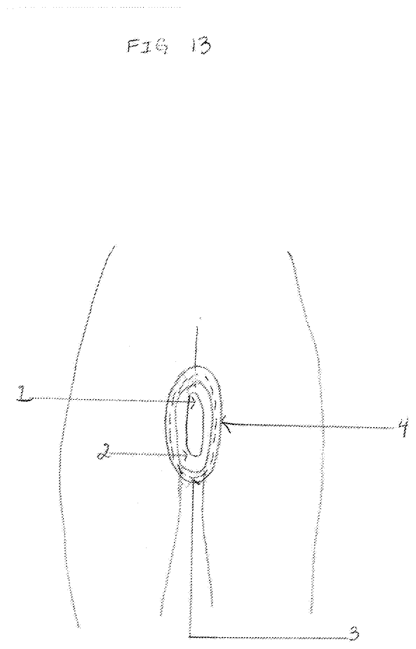

FIG. 13 is a rear view that illustrates how a person can use the invention.

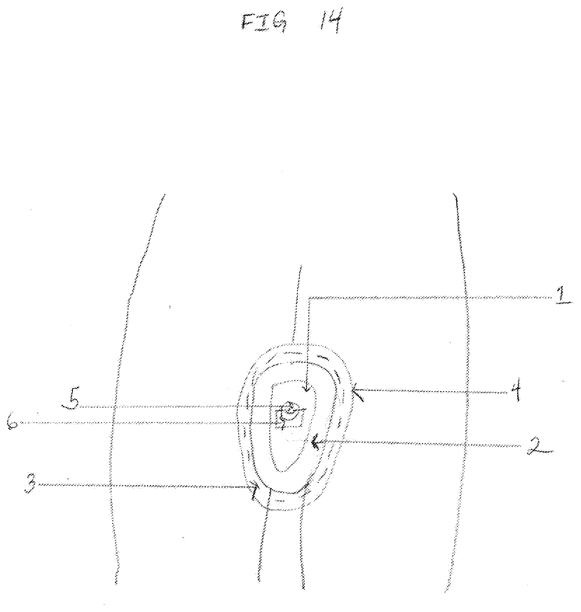

FIG. 14 is a rear view that illustrates how a person can use the invention with the sharp object.

### DETAILED DESCRIPTION OF THE ILLUSTRATED EMBODIMENTS

With reference now to FIG. 1, is a Small Size perspective view of My Shield Device. 1. Is the measured metal which is sanded down and smoothed out on the front, rear, and edges. 2, is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal.

With reference now to FIG. 2, is a Medium Size perspective view of My Shield Device. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal.

With reference now to FIG. 3, is a Large Size perspective view of My Shield Device. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal.

With reference now to FIG. 4, is a frontal elevational view of a Small Size of My Shield Device. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal.

With reference now to FIG. 5, is a frontal elevational view of a Medium Size of My Shield Device. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal.

With reference now to FIG. 6, is a frontal elevational view of a Large Size of My Shield Device. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal.

With reference now to FIG. 7, is a rear elevational view of a Small Size of My Shield Device with a sharp object placed in a metal inset. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal. 5. Is a sharp metal object. 6. Is a metal inset that is welded to the device and made to hold the sharp metal object.

With reference now to FIG. 8, is a rear elevational view of a Small Size of My Shield Device with a sharp object placed in a metal inset. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal. 5. Is a sharp metal object. 6. Is a metal inset that is welded to the device and made to hold the sharp metal object.

With reference now to FIG. 9, is a rear elevational view of a Small Size of My Shield Device with a sharp object placed in a metal inset. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal. 5. Is a sharp metal object. 6. Is a metal inset that is welded to the device and made to hold the sharp metal object.

With reference now to FIG. 10, this is a side view of the small, medium, and large size of My Shield; the opposite side being a mirror image thereof. 1. Is the measured metal. 2. Is the rubber strip. 3. Is the safe adhesive that is placed within the rubber strip.

With reference now to FIG. 11, this is a side view of the small, medium, and large size of My Shield; the opposite side being a mirror image thereof. 1. Is the measured metal. 2. Is the rubber strip. 3. Is the safe adhesive that is placed within the rubber strip. 4, is the metal inset which holds the sharp metal object. 5, is the sharp metal object that is placed within the metal inset.

With reference now to FIG. 12, this a frontal view that illustrates how My Shield is to be worn. It covers the front and goes in between the legs and covers the rear of the person wearing it. This device is sized to be worn by a female, male and children. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal.

With reference now to FIG. 13, this is a rear view without a sharp object that illustrates how My Shield is to be worn. It covers the front and goes in between the legs and covers the rear of the person wearing it. This will be the same for the small, medium, and large size of My Shield. This device is sized to be worn by a female, male and children. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal.

With reference now to FIG. 14, this a rear view with a sharp object that illustrates how My Shield is to be worn. It covers the front and goes in between the legs and covers the rear of the person wearing it. This will be the same for the small, medium, and large size of My Shield. This device is sized to be worn by a female, male and children. 1. Is the measured metal which is sanded down and smooth out on the front, rear, and edges. 2. Is the inside edge of the rubber strip that is placed around the edge of the entire device. 3. Is the safe adhesive that is placed within the rubber strip and placed around the edges of the device. 4. Is the outside edge of the rubber strip that is placed around the edge of the metal. 5. Is a sharp metal object. 6. Is a metal inset that is welded to the device and made to hold the sharp metal object.

### CROSS REFERENCE TO RELATED APPLICATIONS

The application numbers and names are listed below.
875,845 January 1908 Perkins . . . 128/883
1,445,253 June 1915 Rubel . . . 604/394
1,477,187 December 1923 Rayne . . . 602/73 X
2,691,984 October 1954 Zacks . . . 604/394 X
3,227,160 January 1966 Younger . . . 602/70 X
5,174,307 December 1992 Thompson . . . 602/70 X

1. The claimed invention is an up-to-date alternative for a rape device. We would like to make a claim to have the invention patented and make it available commercially. The invention is put together by measured galvanized metal that has been sanded and smoothed down on the front of the metal, on the rear of the metal, and around the edges of the metal. A rubber strip containing a safe adhesive to seal a rubber strip to go around the edge of the metal. My Shield will be made in three different sizes but not limited to the three sizes. It's important to get this patented because it's a simple invention and it provides an up-to-date option to creating a rape device for women, men, and children.

2. My Shield relates to a rape prevention device made with the purpose of preventing or deterring rape. The use of My Shield is to provide an alternative to rape prevention. According to a 2001 World Health Organization study, 20% of women worldwide had been victims of rape or attempted rape at least once in their lives. [3] According to figures from a 2011 report from the Centers for Disease Control and Prevention, one in five women in America are victims of sexual assault. More than 40% of these victims are children, reporting they were first raped before they were 18 years old.

3. Back in the 15^{th} Century, chastity belts were created as a rape prevention device. Although several chastity belts have been invented, none of them have made it to commercial production. My Shield is an easy-to-use invention, and it will be available for women, men, and children. It will be made into three different sizes: small, medium, or large. The wearer will be able to use it to cover the front, middle and rear of their private areas. This invention can be used within their undergarments. The invention can be removed if not needed.
